(19) United States
(12) Patent Application Publication
(10) Pub. No.: US 2002/0091361 A1
(43) Pub. Date: Jul. 11, 2002

(54) Multiple-dose syringe

(76) Inventors: Jack P. Rosoff, Portland, OR (US); Michael N. Hirsch, Portland, OR (US); Ali S. Salem, Canby, OR (US)

Correspondence Address: LeBoeuf, Lamb, Greene & MacRae, L.L.P., Suite 2000, 633 Seventeenth Street, Denver, CO, 80202, US

(21) Appl. No.: 10/093,707

(22) Filed: Mar. 7, 2002

Related U.S. Application Data
(63) Continuation-in-part of application No. 09/923,756, filed on Aug. 6, 2001.
Application No. 09/923,756 is a continuation of application No. 09/392,870, filed on Sep. 9, 1999, now Pat. No. 6,270,482.
(60) Provisional application No. 60/274,669, filed on Mar. 8, 2001.

(51) Int. Cl. A61M 5/24

(52) U.S. Cl. 604/212; 604/232

(57) ABSTRACT

A multiple-dose syringe including a barrel with a closed end and an open end, the closed end having an injection port adapted to receive a needle. A plunger is slidably disposed through the open end of the barrel. A container is connected to an end of the plunger to move with the plunger. The container has a deformable shell with an opening at a forward end thereof and a predetermined quantity of fluid sealed therein by a closure member disposed over the opening. The container is slidably disposed in the barrel and includes a seal proximal to the forward end to form a first cavity in the barrel with a volume that is adjustable by moving the container in the barrel with the plunger so that fluid can be selectively drawn into and expelled from the first cavity. After at least a substantial portion of the fluid is expelled from the first cavity, the shell is configured to be collapsed by further pressure applied by the plunger to expel the predetermined quantity of fluid contained therein.

### REFERENCE TO RELATED PATENT APPLICATIONS

This application claims priority under 35 U.S.C. § 119(e)(1) to U.S. Provisional Application Serial No. 60/274,669 filed Mar. 8, 2001, which is incorporated herein by reference in its entirety, and also claims priority under 35 U.S.C. §120 to co-pending U.S. patent application Ser. No. 09/923,756, filed Aug. 6, 2001, entitled “Multiple-Dose Syringe,” which claims priority as a continuation to U.S. patent application No. 09/392,870, entitled “Multiple-Dose Syringe,” filed Sep. 9, 1999, which issued as U.S. Pat. No. 6,270,482, on Aug. 7, 2001, both of which are incorporated herein by reference in their entirety.

### FIELD OF THE INVENTION

The present invention relates to a syringe, and more particularly, to a syringe adapted to sequentially inject a plurality of fluids.

### BACKGROUND OF THE INVENTION

When administering certain medications, it is sometimes necessary to inject sequentially two fluids into a patient. For example, during chemotherapy, small quantities of medicine are administered, usually through an IV. To insure that all of the medicine reaches the patient, the medication is followed by a saline flush. The saline flush rinses any residual medicant through the IV and into the patient. Traditionally, the saline flush is administered as a separate step from the medicine. In particular, a standard single-dose syringe is used to deliver the medicine. A health care worker then reloads the syringe with the desired quantity of saline. The saline is then injected into the IV to flush the medicine into the patient. This process is time consuming, and, because it requires multiple operations with the needle, it increases the chances health care workers will inadvertently prick themselves with the needle.

Various types of syringes for dispensing sequentially multiple fluid doses have been proposed to address the above problem. For example, U.S. Pat. No. 4,702,737 to Pizzino discloses a multiple-dose, single-barrel syringe utilizing a plurality of telescoping sections of progressively decreasing diameter. Unfortunately, the design of this syringe requires that all of its chambers be pre-loaded with fluids at the time of manufacture. In particular, the syringe incorporates a needle that extends into the barrel of the syringe to puncture a membrane to release the second fluid. The internal needle prevents the syringe from being completely closed to draw fluid into the barrel. As a result of the need to completely preload the syringe, it is necessary to stock separate syringes for each medication. Such medications are often expensive and have limited shelf life, thereby limiting the usefulness of this design.

U.S. Pat. Nos. 4,439,184, 4,715,854, and 5,720,731 to Wheeler, Viallancourt and Armata, respectively, disclose multiple-dose syringes with two pistons and a bypass zone. In each of these patents, a second chamber between the first and second pistons is filled and dispensed through the bypass zone, which is located on one side of the barrel wall near the injection port. Syringes with a bypass zone and multiple pistons are complicated to manufacture and require many specially designed parts. In most of the floating piston designs, the syringe must be preloaded with both fluids because the syringe cannot draw fluids or aspirate. In addition, the floating piston is subject to jamming and may thereby become difficult to depress.

### BRIEF DESCRIPTION OF THE DRAWINGS

FIG. 1 is a side view of a syringe constructed according to the present invention.

FIG. 2 is a side view of the syringe of FIG. 1 with the fluid in a first cavity expelled.

FIG. 3 is a side view of the syringe of FIG. 1 with part of the fluid in a container expelled.

FIG. 4 is a sectional view of a container constructed according to the present invention.

FIG. 5 is an enlarged view of a coupler configured to connect a plunger to the container of FIG. 4.

FIG. 6 is a sectional view of a container cap constructed according to the present invention.

FIG. 7 is an alternative embodiment of the cap of FIG. 6.

FIG. 8 is a block diagram of the steps involved in utilizing the syringe of FIG. 1.

FIG. 9 is a drawing illustrating an alternative embodiment of a multiple-dose syringe with a closure member including a valve that opens in response to pressure.

FIG. 10 is a drawing illustrating the alternative embodiment of a multiple-dose syringe with the valve of the syringe of FIG. 9 in the open state.

FIG. 11 is a more detailed view of an embodiment of the valve of the syringe of FIG. 9.

FIG. 12 is a side view of the valve of FIG. 11.

FIG. 13 is an alternative embodiment of the valve of the syringe of FIG. 9.

### DETAILED DESCRIPTION OF THE INVENTIONS

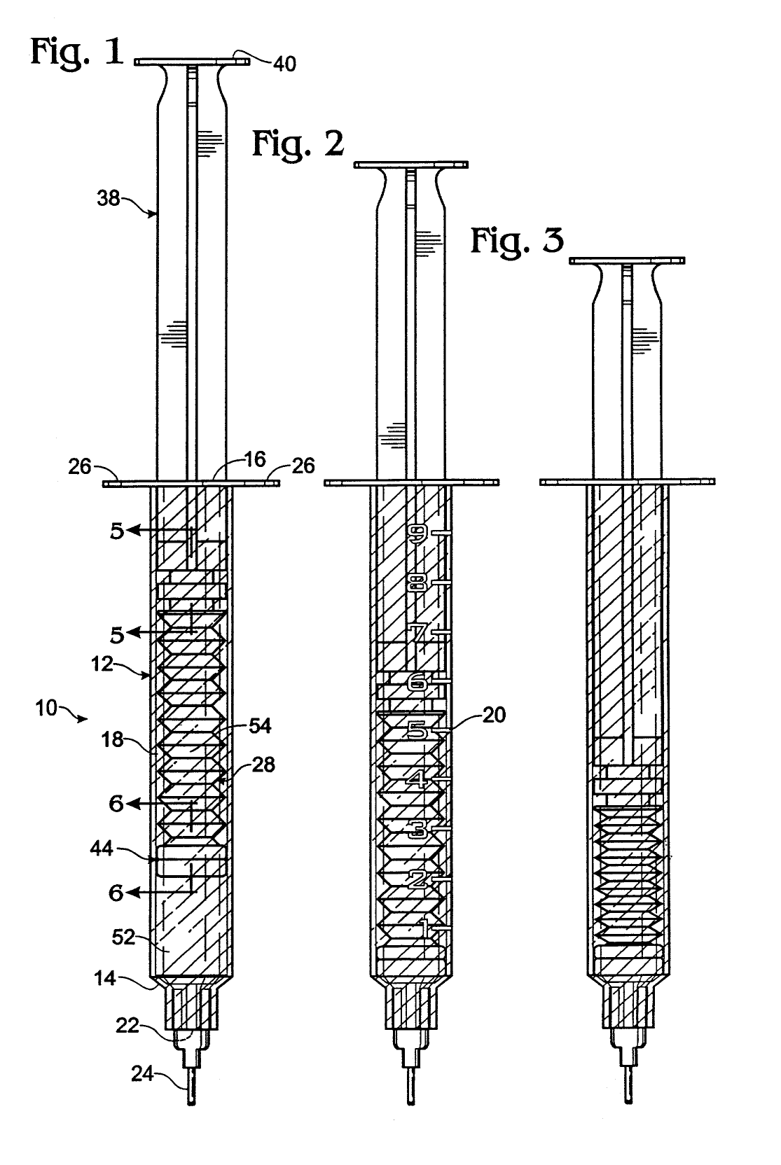

A syringe constructed according to the present invention is shown generally at 10 in FIG. 1. Syringe 10 includes a cylindrical hollow barrel 12 with a closed end 14 and an open end 16. The cylindrical walls of the barrel define a cavity 18, which is adapted to receive and hold the fluid to be dispensed. The cavity typically has a volume or capacity of between 1 and 10 cc, and is marked with gradations 20 to permit the amount of fluid to be measured. It should, or course, be understood, that the present invention could be implemented with syringes of any size. The closed end has an injection port 22 which is configured to receive a needle 24. Finger grips 26 are disposed adjacent to the open end of the barrel and allow the user to grasp the barrel when drawing fluids into or dispensing fluids out of the syringe.

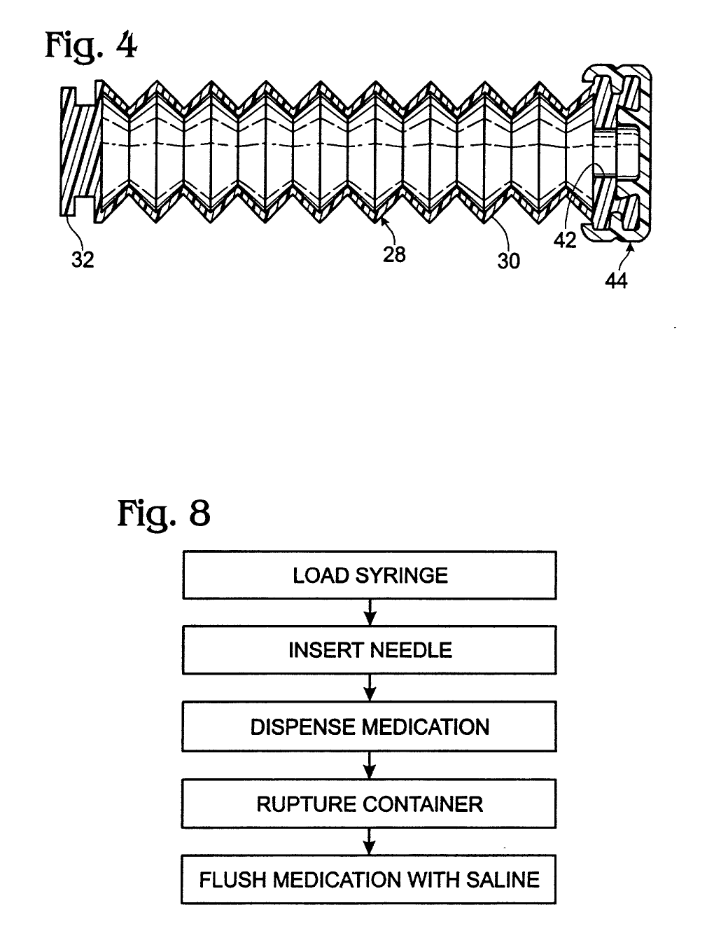

A fluid container 28 is slidably received into barrel 12 through open end 16. As shown in FIG. 4, the container includes a cylindrical bellows-like shell 30. The shell is preferably made of a flexible material that is non-reactive to the fluid stored therein. For instance, polypropylene is a suitable material when the container is used to hold saline. The flexible material allows the container to collapse to dispense fluid, as described in more detail below and illustrated in FIG. 3. It should be understood that other collapsible configurations besides a pleated or bellows structure could be used for shell 30.

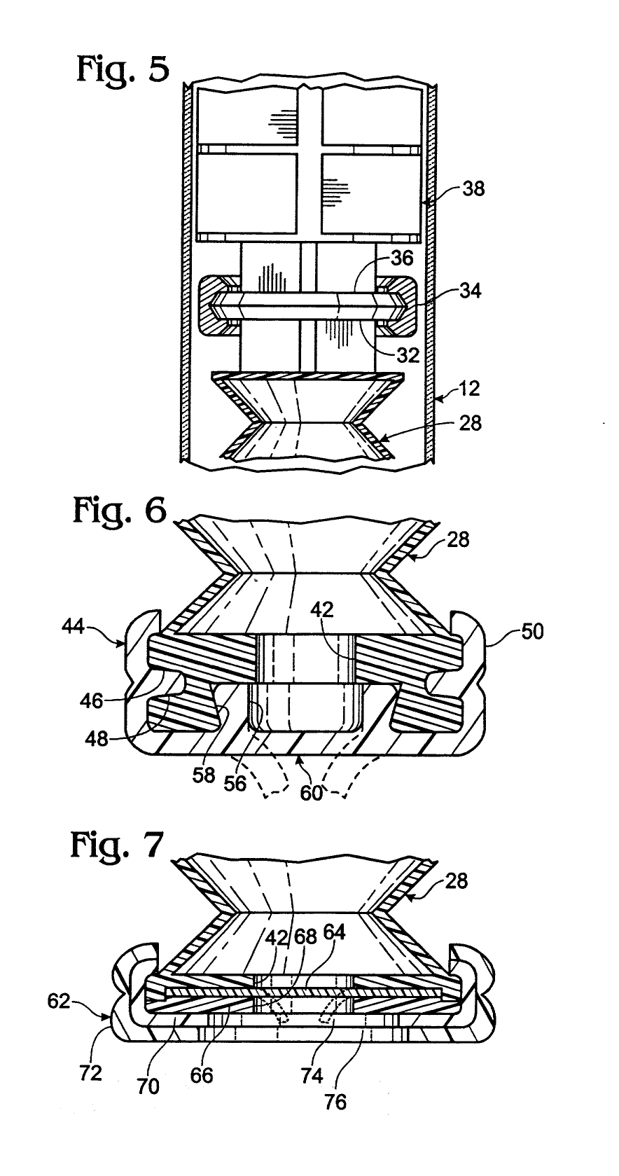

As shown in FIG. 5, a connector 32 is formed on a closed end of the shell. Connector 32 is joined by a coupler 34 to corresponding connector 36 formed on the end of a plunger 38. Plunger 38 has an elongate shaft extending from connector 36 to a thumb pad 40, which is shown in FIG. 1 and is used to depress or retract the plunger. Coupler 34 is preferably formed of a butyl rubber compound and deforms to slip over the connectors. The connection between the container and the plunger allows the plunger to be used to move the container up and down in the barrel. As such, many other connections between the container and the plunger could also be used, including, for instance, glue or clips. Also, the plunger could be formed integrally with the container.

The end of the shell opposite connector 32 includes a passage 42 that is selectively sealed by a closure member in the form of a cap 44, as shown in FIG. 6. The shell includes a circumferential groove 46 that receives a corresponding flange 48 formed on the inside surface of the cap. The cap is preferably formed of a butyl rubber compound to allow it to be fitted over the end of the shell and retained thereon. The outer perimeter of the cap is shaped to form a perimeter seal 50 and sized to fit snuggly within the barrel, similar to the tip on a standard plunger. When the container is placed in the barrel, as shown in FIGS. 1-3, the perimeter seal effectively separates the barrel into two regions or cavities: a first region 52 disposed between the closed end and the cap and a second region 54 disposed behind the cap and occupied by the container.

An inwardly facing cup 56 is formed on the end face of the cap. The walls of the cup are received in a recess 58 formed in the end of the shell proximal to passage 42. A slight outward tilt to the walls of the cup and recess serves to help retain the cap on the end of the shell. In particular, any pressure created in the fluid in the shell tends to urge the walls of the cup outward to tighten the seal between the cap and shell, thereby preventing the escape of fluid and preventing the cap from being pushed off the end of the shell.

The bottom of the cup forms a rupture zone 60 that is pressure rupturable, i.e. ruptures when fluid pressure across the rupture zone exceeds some desired level. For instance, the thickness of the rupture zone may be varied to control the pressure at which rupture occurs. Alternatively, a defect may be created in the rupture zone to provide a predetermined failure location. For example, the defect can be a cut extending part way through the material of the cap or a series of partial perforations. In general, however, the rupture zone should fail at a relatively predictable pressure. Furthermore, the pressure should be readily achievable by finger pressure on the thumb pad of the plunger. It should be noted that any pressure created in the shell is matched by backpressure of the fluid in the first region. Therefore, zone 60 will not rupture until the fluid in the first region is substantially completely expelled. The dashed lines in FIG. 6 depict the rupture zone after rupture.

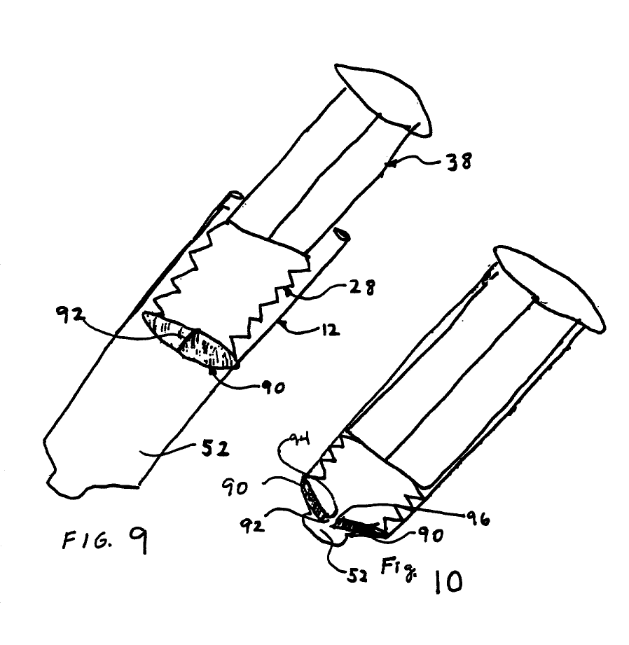

An alternative cap structure 62 is shown in FIG. 7 and includes a rupture sheet 64 disposed over passage 42. The rupture sheet is preferably formed of a thin sheet of rubber, plastic or non-corrosive metal. The rupture sheet is supported and retained against the end of the shell by a seal flange 66 with a central aperture 68 aligned with passage 42. The aperture allows fluid to pass after rupture of the sheet. The seal flange is held in place on the end of the shell by a clamp ring 70 that is crimped over the end of the shell. The clamp ring is preferably formed from a thin deformable cylinder of metal, such as used on the end of a medicine vial. A seal 72, preferably formed of a butyl rubber compound, is disposed over the clamp ring to form a seal with the walls of the barrel, as previously described. The clamp ring and seal include apertures 74 and 76, respectively, that allow fluid from the container to pass after the sheet is ruptured, as shown by the dashed lines in FIG. 6.

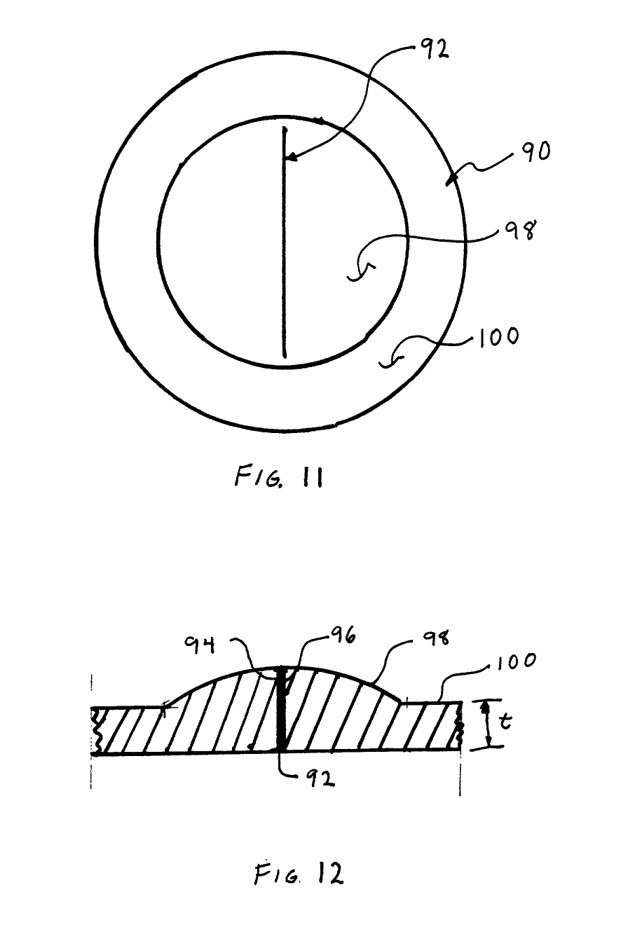

As an alternative embodiment to the closure member in the form of a cap 44 shown in FIG. 6, and as an alternative to the alternative cap structure 62 shown in FIG. 7, FIG. 9 shows the fluid container 28 closed by a closure member including a valve 90. The valve 90 is preferably a valve that opens when the pressure in the container 28 exceeds some desired level (with respect to the pressure in the first region 52). One embodiment of the valve 90 operates much like a “Heimlich”-type valve, which is well known in the medical arts. The “Heimlich” valve was described in U.S. Pat. No. 3,463,159. A “Heimlich” valve consists of a pair of elastic membranes or sheets having a slit between them through which fluids may flow in one direction. This type of valve has been used in a variety of industries, see, e.g., U.S. Pat. No. 4,261,362, and will be readily familiar to a person skilled in the art. A similar valve is found in the well-known “whoopie” cushion found in any toy store. A similar valve has also been referred to as a “condom”-type valve (See U.S. Pat. No. 4,738,672), and could also be used in modified form. The valve 90 may be considered a Heimlich-type valve except that the opposing walls (elements 94 and 96, shown in FIG. 9) of the slit 92 are not as long, i.e., the thickness of valve 90 is not as thick, as the “rubbery tube” of the Heimlich valve shown in the '159 Patent.

In the embodiment shown in FIG. 9, valve 90 consists of a circular elastic membrane with an outer perimeter shaped to form a perimeter seal 50 such as shown in FIG. 6. The valve 90 in FIG. 9 is shown in the closed state. The valve 90 is composed of an elastomer or similar compound, and preferably a butyl or nitryl rubber compound. Other suitable materials will be readily apparent to those skilled in the art. Valve 90 of FIG. 9 includes a slit 92 through the entire thickness (shown, for example, as “t” in FIG. 12) of the valve 90. The slit 92 is held closed by the elasticity of the compound. As noted above, pressure in the container 28 is matched by backpressure of the fluid in the first region 52. Therefore, the valve 90 will not open until the fluid in the first region 52 is substantially expelled, as illustrated in FIG. 10. As shown in FIG. 10, the valve 90 is in the open state, as slit 92 is open, allowing fluid to pass from the container 28 into the first region 52.

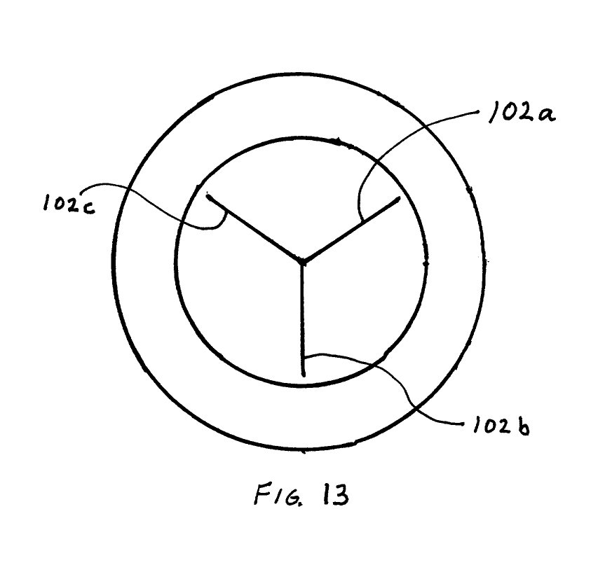

An embodiment of the valve 90 is shown in FIG. 11. It will be apparent to one skilled in the art that the length of the slit 92 and thickness (t, shown in FIG. 12) will depend on the diameter of the container 28. For some sizes, it may be desirable to form a raised region 98 on the outward facing surface 100 (shown in FIG. 11 and FIG. 12). The raised region 98 has a greater thickness than t (shown in FIG. 12). The purpose of the raised region is to provide more surface area for the opposing walls 94 and 96 of the slit 92, thereby providing a better, more positive seal. Other such variations should be apparent to a person skilled in the art.

It should also be understood that the valve 90 may also include a perimeter seal, similar to perimeter seal 50 shown in FIG. 6, and a corresponding flange, similar to flange 48 of FIG. 6, to attach the valve to the container 28. As an alternative, the valve 90 could also be attached to the container 28 as shown in FIG. 7. Alternatively, any other suitable means to attach the valve 90 to the container 28 could be used so long as such means provides a seal between the container 28 and the valve 90 such that fluid substantially only passes through the valve 90, and in the illustrated embodiment, through slit 92. In the embodiments shown in FIGS. 6 and 7, the perimeter seal 52, or alternatively, seal 72, form a seal between the container 28 and the walls of the barrel 12. It should be understood that the valve 90 could be formed to accomplish this function of sealing between the container 28 and the barrel 12, or alternatively, this function could be performed by a separate member. It is contemplated that the plunger 38 and the container 28 could be formed as a single integral component.

FIG. 13 illustrates yet another alternative to valve 90 shown in FIGS. 9-12. FIG. 13 illustrates a valve similar to valve 90 discussed above, except that, instead of a single slit 92, the valve of FIG. 13 has three slits 102a-c of equal length, arranged symmetrically as shown. Alternatively, any number of slits could by used, of the same or different length, arranged symmetrically or asymmetrically. The present invention is not limited to a single slit-type, Heimlich-type, whoopie cushion-type, or condom-type valve, but includes any suitable valve that, when included within a closure member attached to the container 28, operates to allow the fluid to pass from the container when the pressure differential across the closure member, or more precisely, the valve, exceeds some desired level.

As described above, the syringe of the present invention is preferably pre-loaded or filled with saline or other second fluid at the time of manufacture. The plunger is also attached to the container and the resulting assembly is packaged in a sterile condition for shipment. The needle may or may not be attached, depending on the configuration desired. It should be noted that the barrel and plunger of the present invention are preferably unmodified components from a standard syringe design. This eliminates the need to create new and specialized parts for use with a two-fluid syringe. Although it is preferred that the container be pre-loaded in the syringe, it should also be understood that the container could be provided as a separate unit for installation and use with an otherwise standard syringe. This variation is facilitated by use of a design that incorporates unmodified parts from a standard syringe.

FIG. 8 depicts the steps involved in using a syringe according to the present invention. First, the operator selects a pre-loaded syringe package and removes the sterile envelope. If necessary, a needle is attached to the barrel. The operator then loads the desired amount of medicine into the syringe similar to loading a conventional syringe. This is possible because the plunger/container functions like a standard plunger until the medicine in the forward region is expelled. Thus, the operator can retract the plunger to load air into the syringe, insert the needle into a medicine vial, push forward on the plunger to inject the air into the vial and then retract the plunger again to withdraw the desired amount of medicine. The needle is then inserted into an IV, and the medicine is dispensed by depressing the plunger, as shown by comparison of FIGS. 1 and 2. When the medicine is dispensed, subsequent pressure on the plunger causes the cap to open, for example, by rupturing (as illustrated) or by the opening of a valve (for example, the valve of FIGS. 11 or 13), thereby releasing the saline or other fluid in the container. The plunger is then further depressed to compress the container, as depicted in FIG. 3, and force the secondary fluid out, thereby flushing the medicine.

It can be seen that the inventions described herein provide an economical and easy to use solution to the problem of sequentially injecting two fluids. The simple operation saves time and decreases the chances that a health care worker will inadvertently stick themselves with the needle.

The foregoing description of the present invention has been presented for purposes of illustration and description. The specific embodiments thereof as disclosed and illustrated herein are not to be considered in a limiting sense as numerous variations are possible. Applicant regards the subject matter of the invention to include all novel and non-obvious combinations and subcombinations of the various elements, features, functions and/or properties disclosed herein. No single feature, function, element or property of the disclosed embodiments is essential. Consequently, the invention and modifications commensurate with the above teachings and skill and knowledge of the relevant art are within the scope of the present invention. It is intended that the description be construed to include all alternative embodiments as permitted by the prior art.

What is claimed is:

1. A multiple-dose syringe comprising:
a barrel having a closed end and an open end, the closed end having an injection port adapted to receive a needle;
a plunger having a first end and a second end, the second end of the plunger being slidably disposed through the open end of the barrel;
a container having a collapsible shell and having at least a first opening selectively sealed by a closure member including a valve, the container positioned within the barrel with the first opening in proximity to the injection port of the barrel, and
a seal slidably positioned between the container and the barrel.
